SEQUENCE LISTING (3 sequences)

SEQ ID NO: 1 - protein, 258 aa, Artificial Sequence
  Description of Artificial Sequence Synthetic polypeptide
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSGSREFRSYSHGFPPAVAAQDDGTLPMSCAQESGMDRHPAACASARINV

SEQ ID NO: 2 - DNA, 777 bp, Artificial Sequence
  Description of Artificial Sequence Synthetic polynucleotide
atgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttatagccatggcttcccgccggcggtggcggcgcaggatgatggcacgctgcccatgtcttgtgcccaggagagcgggatggaccgtcaccctgcagcctgtgcttctgctaggatcaatgtgtaa

SEQ ID NO: 3 - RNA, 19 nt, Artificial Sequence
  Description of Artificial Sequence Synthetic oligonucleotide
guugcguauuggaagauca

(19) United States
(12) Patent Application Publication
(10) Pub. No.: US 2010/0115635 A1
(43) Pub. Date: May 6, 2010

(54) Methods for Assessing the Delivery of Exogenous Agents

(75) Inventors: Yu Shen, Gurnee, IL (US); Stephen W. Fesik, Nashville, TN (US); Xiaoyu Lin, Gurnee, IL (US); Leiming Li, Buffalo Grove, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: Abbott Laboratories, Abbott Park, IL (US)

(21) Appl. No.: 12/415,610

(22) Filed: Mar. 31, 2009

Related U.S. Application Data
(63) Continuation-in-part of application No. 12/114,609, filed on May 2, 2008.
(60) Provisional application No. 60/916,003, filed on May 4, 2007.

(51) Int. Cl. G01N 33/00; C12Q 1/68; A01K 67/00

(52) U.S. Cl. 800/3; 435/6; 800/13

(57) ABSTRACT

This invention provides methods, compositions and kits for rapid determination of the delivery of exogenous agents both in vitro and in vivo, including without limitation siRNA, microRNA, a ribozyme or an antisense molecule, any of which may target, bind to, or inactivate the mRNA of the gene of interest expressed in the cells. The methods, compositions and kits utilize a promoter-reporter construct whereby successful non-viral nucleic acid delivery leads to an up-regulation of reporter signals thus providing a quantitative, sensitive and rapid means of detection, validation and monitoring.

### RELATED APPLICATION INFORMATION

This application is a continuation in part of U.S. patent application Ser. No. 12/114,609 filed on May 2, 2008, which claims the benefit of U.S. Application No. 60/916,003, filed on May 4, 2007, the contents of each of which are herein incorporated by reference.

### FIELD OF THE INVENTION

The present invention provides methods, compositions and kits for the detection, monitoring and validation of the delivery of an exogenous agent both in vivo and in vitro, utilizing a reporter-promoter system.

### BACKGROUND OF THE INVENTION

RNA interference (RNAi) is a mechanism of post-transcriptional gene silencing using double-stranded RNAs (dsRNAs). The observation that short interfering RNAs (siRNAs) can induce robust gene silencing without triggering interferon response in mammalian cells quickly lead to the adoption of RNAi as a functional genomic tool to study the loss of function phenotypes in mammalian systems. In addition to RNAi's ability to be utilized as a research tool, it has been thought that siRNAs also hold promise as novel therapeutic agents.

Recently, though, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. (2001) Nature 411: 494-498). These RNA duplexes are too short to elicit sequence-nonspecific responses, yet they efficiently initiate RNAi. This was a stunning discovery and many laboratories around the country immediately rushed to have siRNA made to knock out their favorite gene in mammalian cells. The results demonstrate that siRNA appears to work quite well in most instances, far better and more consistently than do ribozymes, antisense or other nucleic acid agents.

However, a major limitation to the use of siRNA in mammals is the method of in vivo delivery. Although conventional methods such as Western, PCR and Northern analyses, can be used to monitor siRNA delivery to tumors, these methods often fail to produce reliable results unless robust target knockdown occurs in a large percentage of cells in the targeting tissue. Therefore, the ability to identify, compare and validate whether the siRNA has been delivered into the tissues and has suppressed the target gene expression is crucial. Moreover, there are currently no reliable assays available that evaluate, compare and optimize various delivery approaches of in vivo delivery. Accordingly, there is now a need for a rapid, reliable and sensitive method of detection, validation and monitoring of an exogenous agent and preferably siRNA delivery both in vitro and in vivo.

### SUMMARY OF THE INVENTION

The present invention relates to methods, compositions and kits for the detection and characterization of delivery of exogenous agents, such as small interfering RNAs (RNAi) and other short nucleic acid molecules. More particularly, the present invention relates to improved methods for the quantitative detection of the delivery into a cell of short RNAs containing fewer than 22-25 nucleotides in vivo and in vitro.

The invention further provides a destabilized tetracycline (tet) repressor protein (tetR-ODC) with a half-life of about 72 hours or less, preferably 12 hours or less; most preferably about 4 hours or less, and even more preferably about 2 hours or less.

Preferably, the composition and methods of the present invention include a promoter having binding sites for destabilized tet repressor protein (tetR-ODC) as well one or more indictors or reporters of cellular delivery of the agent in vitro and in vivo, and preferably, the reporter detects via enzymatic activity.

Thus, the present invention is directed to a fusion protein comprising a tet repressor so that the resulting fusion protein has a half-life of no more than about 48 hours and as little as less than one hour. In a preferred embodiment, the tetR fusion protein comprises fusion to PEST sequence containing portion of a C-terminus of murine ornithine decarboxylase (MODC). One example of the tetR fusion protein of the present invention has the sequence shown in SEQ ID NO: 1.

The present invention is also directed to an isolated DNA molecule encoding the fusion protein, which comprises a tetR protein. One example of the isolated DNA of the present invention has the sequence shown in SEQ ID NO: 2. The present invention is also directed to a vector capable of expressing this isolated DNA molecule. The present invention is also directed to a method of producing a stable cell line that upon contacting an exogenous agent, expresses a detectable protein, e.g., beta galactosidase encoded by the bacterial gene lacZ, comprising the step of transfecting cells with a vector disclosed herein.

Accordingly, the exogenous agent, which may be used within the scope of the invention, may be a nucleic acid molecule such as a short interfering RNA, microRNA, a ribozyme or an antisense molecule, any of which may target, bind to, silence, or inactivate the mRNA of the gene of interest expressed in the cells. Such nucleic acid molecules may be introduced into the cells by a variety of ways. The exogenous agents may further be complexed with at least one additional molecule which enables the delivery of the agent into a cell for therapeutic administration, such molecules may include but are not limited to, encapsulating substances, cholesterol moieties, cationic lipids, inclusion complexes such as for example linear cyclodextrin copolymers and linear oxidized cyclodextrin copolymer, a dendrimer (i.e., a highly branched polymers with well-defined architecture), biodegradable targetable microparticle delivery systems, as well as nanoparticles. Additionally, the exogenous agent can be included in a lipid based formulation. Examples of a lipid based formulation include, but are not limited to, 1,2-dioleoyl-3-trimethylammonium-propane, 1,2-dioleoyl-3-(dimethylamino) propane, 3β-[N-(N′,N′-dimethylaminoethane)-carbamoyl]cholesterol, cholesterol, dioleoyl phosphatidylethanolamine, 1,2-distearoyl-sn-glycero-3-phosphoethanolamine-N-(carboxy(polyethylene glycol)2000) or combinations thereof.

In yet a further embodiment, there is provided a method for analyzing the delivery of an exogenous agent to a tissue comprising: (a) providing a tissue; (b) contacting the tissue with the exogenous agent; (c) analyzing a sample from the tissue, thereby providing analysis of the delivery of the exogenous agent to the tissue. Preferably said tissue comprises the tetR fusion protein comprises fusion to PEST sequence containing portion of a C-terminus of murine ornithine decarboxylase (MODC). One example of the tetR fusion protein of the present invention has the sequence shown in SEQ ID NO: 1. The exogenous agent can be included in a lipid based formulation. Examples of a lipid based formulation include, but are not limited to, 1,2-dioleoyl-3-trimethylammonium-propane, 1,2-dioleoyl-3-(dimethylamino) propane, 3β-[N-(N′,N′-dimethylaminoethane)-carbamoyl]cholesterol, cholesterol, dioleoyl phosphatidylethanolamine, 1,2-distearoyl-sn-glycero-3-phosphoethanolamine-N-(carboxy(polyethylene glycol)2000) or combinations thereof.

Another embodiment of the invention is directed to a kit for in vitro and/or in vivo gene knockdown studies at a RNA level which includes the following parts: 1) providing a tissue (or other population of cells) having the tetR ODC reporter fusion protein which produces a signal, e.g., light, e.g., fluorescence, which is correlated with the delivery and of the exogenous agent, e.g., gene which produced short interfering RNA (siRNA) against tetR; and 2) evaluating a signal produced by the reporter agent (e.g., the production of light, e.g., fluorescence) that exhibits reporter gene activation if it is successfully delivered the agent.

Yet another aspect of the invention related to a transgenic non-human animal containing a recombinant nucleic acid molecule stably integrated in its genome, where the recombinant nucleic acid molecule encoding the fusion protein, which comprises a tetR protein. In a preferred embodiment, the tetR fusion protein comprises fusion to PEST sequence containing portion of a C-terminus of murine ornithine decarboxylase (MODC). One example of the tetR fusion protein of the present invention has the sequence shown in SEQ ID NO: 1.

The invention also related to a method of detecting delivery into a cell of an exogenous agent, including the steps of (a) administering an exogenous agent, preferably a tetR siRNA to a non-human transgenic animal containing a recombinant tet-responsive LacZ reporter tetR fusion protein which further comprises fusion to PEST sequence containing portion of a C-terminus of murine ornithine decarboxylase (MODC); and (b) measuring with a photodetector device, photon emission through the tissue, thereby detecting though the silencing of the tetR delivery of the exogenous agent into the cell.

A transgenic non-human animal comprising a recombinant nucleic acid molecule stably integrated into the genome of said animal, said molecule encoding a tet fusion protein further comprises a PEST sequence, wherein said PEST sequence is a PEST sequence-containing portion of a C-terminus of murine ornithine decarboxylase (MODC) fused to a detectable marker. Wherein the detectable marker is selected from the group consisting of humanized renilla green fluorescent protein (hrGFP), enhanced green fluorescent protein (eGFP), enhanced blue florescent protein (eBFP), enhanced blue fluorescent protein (eBFP), enhanced cyan fluorescent protein (eCCFP), enhance yellow fluorescent, red fluorescent protein (RFP or DsRed), beta-galactosidase, and luciferase.

A method of analyzing the delivery of an exogenous agent to a tissue comprising: (a) providing a non-human transgenic non-human animal comprising a recombinant nucleic acid molecule stably integrated into the genome of said animal, said molecule encoding a tet fusion protein further comprises a PEST sequence, wherein said PEST sequence is a PEST sequence-containing portion of a C-terminus of murine ornithine decarboxylase (MODC) fused to a detectable marker; (b) contacting said non-human animal with the exogenous agent; (c) analyzing a sample from the tissue of the non-human animal, thereby providing analysis of the delivery of the exogenous agent to the tissue, wherein said detectable marker is beta-galactosidase. The exogenous agent can be included in a lipid based formulation. Examples of a lipid based formulation include, but are not limited to, 1,2-dioleoyl-3-trimethylammonium-propane, 1,2-dioleoyl-3-(dimethylamino) propane, 3β-[N-(N′,N′-dimethylaminoethane)-carbamoyl]cholesterol, cholesterol, dioleoyl phosphatidylethanolamine, 1,2-distearoyl-sn-glycero-3-phosphoethanolamine-N-(carboxy(polyethylene glycol)2000) or combinations thereof.

A method of analyzing the delivery of an exogenous agent to a tissue comprising: (a) providing a non-human transgenic non-human animal comprising a recombinant nucleic acid molecule stably integrated into the genome of said animal, said molecule encoding a tet fusion protein further comprises a PEST sequence, wherein said PEST sequence is a PEST sequence-containing portion of a C-terminus of murine ornithine decarboxylase (MODC) fused to a detectable marker; (b) contacting said non-human animal with the exogenous agent; (c) non-invasively detecting the delivery of the exogenous agent to the tissue wherein said non-invasive detection is by an imaging, and more specifically by bioluminescence imaging, and wherein the detectable marker is luciferase. The exogenous agent can be included in a lipid based formulation. Examples of a lipid based formulation include, but are not limited to, 1,2-dioleoyl-3-trimethylammonium-propane, 1,2-dioleoyl-3-(dimethylamino) propane, 3β-[N-(N′,N′-dimethylaminoethane)-carbamoyl]cholesterol, cholesterol, dioleoyl phosphatidylethanolamine, 1,2-distearoyl-sn-glycero-3-phosphoethanolamine-N-(carboxy(polyethylene glycol)2000) or combinations thereof.

### BRIEF DESCRIPTION OF THE DRAWINGS

The application file contains at least one drawing executed in color. Copies of this patent with color drawing(s) will be provided by the Patent and Trademark Office upon request and payment of the necessary fee.

FIG. 1A illustrates the principle of the tetR-based positive readout system for monitoring functional siRNA delivery.

FIG. 1B illustrates the tetR reporter promoter system for assessing and validating siRNA delivery efficacy in vivo and particularly in various regions of tumor tissues to create tumor models. In one particular embodiment of the present invention, siRNA is delivery to tumors and can be monitored using, for example IHC or enzymatic detection such as for example, detection via beta-galactosidase expression.

FIG. 2A shows a wild type tetR protein with a half-life far longer than 72 hours, demonstrating that a system based on the wild type tetR protein may not respond promptly to the delivery of the exogenous agent, such as for example siRNA.

FIG. 2B shows the tetR-ODC protein exhibiting a reduced half-life of approximately 2 hours.

FIG. 2C illustrates the full activity of the tetR-ODC compared to the wild type tetR as shown in transfected H1299 cells, using a tet-responsive lacZ reporter construct together with the wild-type tetR and the tetR-ODC expression construct and monitoring the transfected cells of their responses to doxycycline treatment. Cells transfected with the wild type tetR or the tetR-ODC expression construct exhibited similar levels of basal and doxycycline-induced beta galactosidase activity, demonstrating that tetR-ODC retains the full activity of the wild type tetR.

FIG. 3A shows the ability of different tetR-ODC short hairpin RNAs (shRNAs) to knockdown the tetR-ODC protein.

FIG. 3B shows the ability of a tetR-ODC shRNA to regulate the tet-responsive LacZ reporter using a co-transfection assay.

FIG. 4A demonstrates the induction of beta galactosidase activity by doxycycline in MDA-MB435SLM-derived clones with stablely integrated tetR-ODC expression cassette and the tet-responsive LacZ reporter.

FIGS. 4B and 4C demonstrates the induction of beta galactosidase activity by a tetR siRNA in MDA-MB435SLM-derived clones with integrated tetR-ODC expression cassette and the tet-responsive LacZ reporter. The beta galactosidase assay was used in FIG. 4B and the X-gal staining was used in FIG. 4C.

FIGS. 5A, 5B and 5C demonstrates the induction of beta galactosidase activity by doxycycline in flank tumors using the following assays: whole mount X-gal staining (5A), beta galactosidase assay using tumor lysate (5B), and immunohistochemistry-based detection of beta galactosidase in tumor sections using an beta galactosidase antibody (5C).

FIG. 5D shows the induction of beta galactosidase activity by doxycycline in orthotopic liver tumors using whole mount X-gal staining.

FIG. 6A demonstrates the induction of luciferase activity by doxycycline in orthotopic liver tumors using bioluminescence imaging.

FIG. 6B demonstrated in vivo characterization of a delivery system for delivering siRNA into the liver tumor using bioluminescence imaging in the tet-responsive luciferase reporter model.

### DETAILED DESCRIPTION OF THE INVENTION

### I. General

This invention provides a novel system for rapid determination, validation and monitoring of exogenous agents, particularly nucleic acid function in vivo and in vitro with high sensitivity and reliability. The system provides a robust, convenient and reliable platform for evaluating various delivery systems for their efficiency in delivering these exogenous agents, preferably nucleic acid, and most preferably RNAi, into the cell, utilizing a promoter-reporter system.

Generally, a promoter-reporter system is a recombinant polynucleotide inside or outside the cell, in which a promoter is operatively linked to a reporter gene. A promoter-reporter cell is a cell genetically altered (either stably or transiently) so as to contain a promoter reporter construct. In the case of the present invention, the presence of a transcriptional repressor prevents the reporter gene expression, and the effective delivery of an exogenous agent silences the repressor, in turn causing the expression of the reporter gene in the cell.

Typically, a promoter is a DNA sequence involved in initiating transcription of the encoding region of a gene to which it is linked. It may cause constitutive expression of the gene; it may be upregulated in a tissue-specific way; or it may be upregulated in response to efficient delivery of a nucleic acid, as is the case in the present invention. Furthermore, reporter genes are typically any nucleic acid sequence which, when expressed in a cell, causes the cell to display a detectable label, such as a fluorescent or phosphorescent signal, a protein, or enzyme activity detectable in an assay or an antigen detectable on or in the cell by a specific stain, antibody or lectin.

Particularly, the system of the present invention utilizes a repressor-based inducible expression system, whereby a rapid turnover of a destabilized repressor and preferably a tetR repressor, can be used. Such applications include using the destabilized repressor, and preferably a tetR-based inducible system, engineered so that binding of the tetR homodimers to tet operators impedes the binding of TATA binding protein (TBP) and other accessory proteins to the promoter, thereby inhibiting transcription. When the tetR protein binds to the inducing agent, tetracycline/doxycycline, it undergoes conformational changes that abolish the tet operator binding property to tetR, allowing expression from the tet-responsive promoter. As such, when the agent, preferably a nucleic acid and most preferably RNAi, against tetR, is introduced into cells, it serves as an inducing agent by knocking down tetR. As such, one embodiment of the present invention provides a tetR-based positive-readout system that exhibits rapid and robust responses to siRNA delivery in cultured cells in vitro and in vivo.

In accordance with the present invention the tet repressor refers to a prokaryotic protein which binds to a tet operator sequence in the absence but not the presence of tetracycline. The term “tet repressor” is intended to include repressors of different class types, e.g., class A, B, C, D or E tet repressor.

As such, the efficacy of an agent, preferably a nucleic acid and most preferably a siRNA against tetR, is delivered into the cell, via any means, the efficacy of the delivery is determined by its ability to silence the repressor and consequently activate the expression of the reporter gene. If the agent, preferably a nucleic acid is effectively delivered and targets and inactivated expression of its target gene, preferably the destabilized tet repressor (tetR-ODC), a marked increase in reporter expression, for example beta-galactosidase enzymatic activity is observed; and conversely if it fails to be delivered into the cell and knockdown its target gene, a significant change in reporter expression is not observed. Both of these activities are subject to quantitation.

The quantitative silencing effect of an effective delivery of the exogenous agent is determined by an in vitro method using a recombinant tetR reporter gene promoter activity system. Reporter genes for use in the invention encode detectable proteins, which include, but are by no means limited to, chloramphenicol transferase (CAT), beta-galactosidase (beta-gal), luciferase, green fluorescent protein (GFP) and derivatives thereof, yellow fluorescent protein and derivatives thereof, alkaline phosphatase, other enzymes that can be adapted to produce a detectable product, and other gene products that can be detected, e.g., immunologically (by immunoassay).

A screen according to the invention involves detecting expression of the reporter gene by the host cell when contacted with the exogenous agent. If there is no change in expression of the reporter gene, the agent has not been effectively delivered. If reporter gene expression is modified, in particular enhanced, the delivery of the exogenous agent is effective, validated and monitored.

The present invention also embodies an effective reliable and robust way of assessing the delivery of an exogenous agent, preferably nucleic acid delivery, and most preferably RNAi delivery in vivo.

Typically, the difficulties associated with the development of siRNA therapy is the lack of reliable assays that would allow comparing and optimizing various delivery approaches in vivo, i.e., the testing of siRNA delivery in tissue, and preferably in xenograft tumors. Although it is theoretically possible to monitor siRNA delivery to tumors using for example, Immunohistochemistry (IHC), Western, PCR, or Northern analyses, these methods often fail to produce reliable results unless robust target knockdown occurs in the majority of tumor cells. As such, another embodiments of the present invention utilize a repressor-based inducible expression system, such as for example the tetR system, to monitor and validate agent, preferably siRNA, mediated target knockdown and in turn provide a reliable positive-readout assays in which successful siRNA delivery leads to an up-regulation of reporter signal.

### II. Destabilized Repressor of a Repressor-Regulated Promoter

The present invention includes a destabilized repressor and preferably a destabilized tetracycline/doxycycline (“tet”) repressor protein, having a rapid turnover in a cell. More specifically, this destabilized repressor comprises a fusion protein, which has a half-life of no more than about 72 hours, preferably no more than about 48 hours, most preferably with a half-life of no more than 12 hours, and even more preferably a half-life of about 2 hours. In one embodiment, the engineered repressor is tetR fusion protein of tet and a peptide that inclusion of which produces a destabilized protein. An example of such a peptide is the PEST domain of murine ornithine decarboxylase (ODC). In an illustrative case, the PEST domain of ODC from amino acids 422 to 461 was appended to the C-terminal end of a tetR protein (tetR-ODC). The half-life of the tetR-ODC fusion protein was about 2 hours, while that of wild type tetR was more than 72 hours. The ornithine decarboxylase degradation domain dramatically decreases tetR stability.

The rapid turnover version of the repressor and preferably the tet repressor, has a number of advantages over wild type repressors. One such example is that the destabilized tetR-ODC decreases accumulation of tetR. Accumulation of tetR protein can interfere with the sensitivity of analysis. Thus, the destabilized, rapid turnover fusion protein renders more sensitive results and more rapid response. Moreover, having a repressor with a shorter half life can be used to study processes such as for example siRNA delivery both in vivo and in vitro.

Furthermore, the activity of the tetR-ODC functions substantially similar to that of the wild type tetR protein.

### III. siRNA Against TetR

The methods of the present invention typically involves silencing or inhibiting a gene of interest expressed in the cells, such as for example the inhibiting or knockdown of the tetR. Particularly, it is the interaction between the nucleic acids and the cell, either in vitro, such as for example in cultured cells, or in vivo, such as for example in xenograft tumors, and typically will involve inhibiting the binding of a repressor to the promoter, thereby activating transcription. Preferably, the nucleic acid being introduced into the cell is a tetR siRNA, and most preferably complexed with at least one or more molecules, which can be used to transfer therapeutic exogenous agents, and preferably siRNA, into cells.

The exogenous agents in accordance with the present invention may be short double stranded nucleic acid duplexes comprising annealed complementary single stranded nucleic acid molecules, including without limitation siRNA, microRNA, short hairpin RNA (shRNA), RNAs produced by processing of shRNAs, a ribozyme or an antisense molecule, any of which may target, bind to, or inactivate the mRNA of the gene of interest expressed in the cells, preferably tetR.

There is no particular limitation in the length of the short RNA molecules that can be characterized and quantitated by the method of this invention. Short RNAs can be, for example, 17 to 49 nucleotides in length, preferably 17 to 35 nucleotides in length, and are more preferably 17 to 29 nucleotides in length. The short RNAs may contain double-stranded RNA portions where such portions are completely homologous, contain non-paired portions due to sequence mismatch (the corresponding nucleotides on each strand are not complementary) or the short RNAs may contain a bulge (lack of a corresponding complementary nucleotide on one strand), and the like.

In preferred embodiments, the siRNAs are short double stranded RNAs comprising annealed complementary single strand RNAs. Most preferably, each single stranded nucleic acid molecule of the siRNA duplex is of from about 21 nucleotides to about 27 nucleotides in length. In preferred embodiments, duplexed siRNAs have a 2 or 3 nucleotide overhang with 5′ phosphate and 3′-hydroxyl groups.

However, the invention also encompasses embodiments in which the siRNAs comprise an annealed RNA:DNA duplex, wherein the sense strand of the duplex is a DNA molecule and the antisense strand of the duplex is a RNA molecule (e.g., DNA and RNA of less than, for example, 40, 30, 20 or 15 nucleotides in length).

Most preferably the exogenous agent introduced into the cell is a tetR siRNA in complex with at least one additional molecule, carrier and/or complexing agent used to delivery the exogenous agent as for example a therapeutic.

The RNAi according to the invention, preferably contain nucleotide sequences that are identical to a portion of preferably the engineered tetR-ODC gene or its 3′ untranslated regions (3′ UTR). However, RNA sequences with insertions, deletions, and single point mutations relative to the target sequence have also been found to be effective for RNAi mediated inhibition of target gene expression (see, e.g., U.S. Pat. No. 6,506,559). Therefore, 100% sequence identity between the siRNA and the target gene is not required to practice the invention. As such, RNAi agents with insertions, deletions and/or single point mutations relative to the target sequence may also be used in accordance with the present invention.

The degree of sequence identity between a RNAi and its target gene may be determined by sequence comparison and alignment algorithms known in the art (see, for example, Gribskov and Devereux Sequence Analysis Primer (Stockton Press: 1991). The percent similarity between the nucleotide sequences may be determined, for example, using the Smith-Waterman algorithm as implemented in the BESTFIT software program using default parameters. Greater than 90% sequence identity between the RNAi and the portion of the target gene corresponding to the RNAi is preferred.

### IV. Reporter Genes

To detect potential up or down-regulation of the activity of a promoter such as for example the tet-responsive promoter that regulated by the tetR-ODC protein, it is operably linked to a reporter gene that generates a detectable signal.

The reporter gene can encode a protein that produces a fluorescent or phosphorescent signal when expressed in the cells. In this way, behavior of the promoter can be measured in situ. Autofluorescent proteins can be selected from humanized renilla green fluorescent protein (hrGFP), enhanced green fluorescent protein (eGFP), enhanced blue florescent protein (eBFP), enhanced blue fluorescent protein (eBFP), enhanced cyan fluorescent protein (eCCFP), enhance yellow fluorescent, or red fluorescent protein (RFP or DsRed). Bioluminescent proteins include firefly luciferase, Rennilla luciferase and luciferase form the nematode. Enzymes that can be used to convert chemoluminescent substrates include alkaline phosphatase, peroxidase, chloramphenicol acetyl transferase, and beta-galactosidase (encoded by the bacterial gene lacZ).

Also contemplated are reporters that generate a signal detectable by other means. Exemplary are genes which when expressed cause release of a biomolecule into the medium, or cause catalysis of a substrate in the medium into a detectable product. Regulation of the promoter can be followed by assaying the biomolecule or the catalyzed product in the culture supernatant, for example, by immunoassay.

### V. Cell Lines—In Vitro

The cells of this invention are designed to have one or more reporter genes expressed under the control of a promoter or other transcription regulator sequence that responds to a change in the cultural environment, preferably the silencing of a target gene by the effective delivery of the agent. The reporter gene refers to a nucleic acid comprising a nucleotide sequence encoding a protein that is readily detectable either by its presence or activity, including, but not limited to, luciferase, fluorescent protein (e.g., green fluorescent protein), chloramphenicol acetyl transferase, beta-galactosidase, secreted placental alkaline phosphatase, beta-lactamase, human growth hormone, and other secreted enzyme reporters. Generally, a reporter gene encodes a polypeptide not otherwise produced by the host cell, which is detectable by analysis of the cell(s), e.g., by the direct fluorometric, radioisotopic or spectrophotometric analysis of the cell(s) and typically without the need to kill the cells for signal analysis. In certain instances, a reporter gene encodes an enzyme, which produces a change in fluorometric properties of the host cell, which is detectable by qualitative, quantitative, or semiquantitative function or transcriptional activation. Exemplary enzymes include esterases, beta-lactamase, phosphatases, peroxidases, proteases (tissue plasminogen activator or urokinase), and other enzymes whose function can be detected by appropriate chromogenic or fluorogenic substrates known to those skilled in the art or developed in the future.

As such once a promoter-reporter system has been selected, the cells are genetically altered by standard recombinant techniques to place the reporter gene under the control of the promoter. A cell is said to be “genetically altered,” or “trasfected” when a polynucleotide has been transferred or delivered into the cell or tumor by any suitable means of artificial manipulation. This can be done by transfecting the cells with a vector wherein the promoter and reporter are both heterologous to the cell and already linked as an expression cassette. The cassette can then be placed into the genome in a random fashion. Alternatively, the cassette can be placed into a defined locus in the genome by homologous recombination. This has the advantage of placing the promoter into the location in the genome known to be permissive for transcription under appropriate circumstances.

In one particular embodiment, the cell lines may be engineered to respond to the delivery of the tetR-ODC siRNA with an increase of the reporter, preferably beta-galactosidase, which in turn provides the detection of the lacZreporter activity (MDA-tetR-ODC/LacZ cells).

In yet another embodiment of the present invention, the cell lines may be engineered to respond to the delivery of the tetR-ODC siRNA with an increase of the reporter, preferably tet-responsive firefly luciferase reporter, which can be quantitatively assessed, by for example a non-invasive detection method, preferably by an imaging approach, and more preferably by bioluminescence imaging. The cell lines specifically engineered to respond to the delivery of the tetR-ODC siRNA and bioluminescence imaging using the luciferase reporter activity of the present invention is MDA-tetR-ODC/Luc cells.

### VI. Tumors—In Vivo

Repressor-based inducible expression systems such as the tetR system may be adapted to establish the positive-readout assay for the ability of the exogenous agent, preferably siRNA, to be delivered to and persist in a target tissue, by assessing target knockdown in tissue. In addition, it may be desirable to assess the ability of the agent to create another non-endogenous product, such as a protein expressed from an expression vector, a drug created from a prodrug, or a product created from an enzyme.

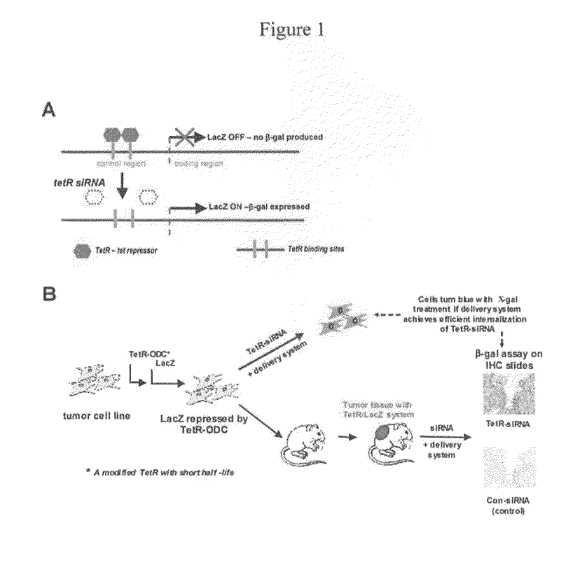

In the tetR-based inducible system, two tet operators are engineered into the CMV promoter downstream of the TATA box. Binding of the tetR homodimers to tet operators impedes the binding of TATA binding protein and other accessory proteins to the promoter, thereby inhibiting transcription. When the tetR protein binds to the inducing agent tetracycline/doxycycline, it undergoes conformational changes that abolish the tet operator binding property of tetR, allowing expression from the tet-responsive promoter. It is conceivable that siRNAs against tetR, when introduced into cells, could also serve as an inducing agent by knocking down the tetR protein (FIG. 1A). The tetR-based positive readout system can be readily introduced into tumor cell lines to create tumor models, and siRNA delivery to tumors can be monitored using IHC or enzymatic detection of beta-galactosidase expression (FIG. 1B).

In accordance with the present invention, the tissue may be an intact tissue or may be samples extracted from the tissue. The tissue samples are obtained by standard methodologies.

The tissue may be animal tissue, for example, liver tissue, lung tissue, prostate tissue, breast tissue, colon tissue, skin tissue or tissue that contains body fluids or contains traces of such fluids such as blood, CSF, urine, saliva, mammary fluid. The animal tissue may be diseased or injured, such as cancerous, inflamed, infected, congenitally diseased, functionally compromised (diabetes, neurodegenerative, or atrophy), traumatized or environmentally insulted.

The tissue may be treated in order to liberate proteins for further analysis. A wide variety of techniques may be applied including, for example, detergent extraction.

In yet a further embodiment of the present invention is creating non-human transgenic animals integrated with the tetR-ODC system. Such transgenic animals may be obtained, for example, by injecting the polynucleotide into a fertilized egg which is allowed to develop into an adult. Such non-human transgenic animals can be used, for example, in screening assays designed to identify the positive delivery of the exogenous agent, preferably siRNA.

Particularly, the high throughput and highly quantitative assay for in vivo characterization of exogenous delivery into an animal and particularly into the tumor of a non-human animal utilizes non-invasive visual detection means and preferably by imaging and more preferably by florescence and bioluminescence (also called chemiluminescence) imaging. Since bioluminescent signals can be acquired in live animals, the imaging-based method eliminates time-consuming steps, such as for example, tumor collection, section and staining) that is vital for an immunohistochemistry-based approach. The higher throughput and better quantification of the imaging-based method further allows for in vivo SAR analysis using a set of variants derived from the most promising delivery system. Considering the lack of correlation between in vivo target knockdown and in vitro transfection observed using many delivery systems, the capability of in vivo SAR analysis will be critical for any chemistry effort aimed at optimizing a promising delivery system.

Bioluminescence imaging requires a reporter construct to effect production of a protein, preferably luciferase, an enzyme that provides imaging contrast by the light emission that results from the luciferase-catalyed conversion of D-luciferin to oxyluciferin. Luciferase is but an example of a light emitting reported, used in the imaging system, other light emitting reports having a characteristic wavelength of light emission, and optimal parameters as well as minimal interference, know to one skilled in art, can be used in the present invention. Furthermore, other luciferases besides the firefly variety including Renilla luciferase, and luciferase form nematode, for example, can also be used in the imaging process of the present invention. Moreover, more than one light emitting reported can be imaged simulatenously in the present invention.

A transgene is a construct that has been or is designed to be incorporated into the cell, e.g., a mammalian cell, that is incorporated in a living animal which that the construct containing the nucleotide sequence, preferably the tetR-ODC protein, is expressed.

A present transgenic non-human animal can be, e.g., a mammal, a bird, a reptile or an amphibian. Suitable mammals for uses described herein include: rodents; ruminants; ungulates; domesticated mammals; and dairy animals. Preferred animals include: rodents, goat, sheep, camels, cows, pigs, horses, oxen, llamas, chickens, geese, and turkeys. In a preferred embodiment, the non-human animal is a mouse or a rat.

Various methods for producing transgenic animals have been described (see, e.g., Watson, J. D., et al., “The Introduction of Foreign Genes Into Mice,” in Recombinant DNA, 2d Ed., W.H. Freeman & Co., New York (1992), pp. 255-272; Gordon, J. W., Intl. Rev. Cytol. 115:171-229 (1989); Jaenisch, R., Science 240:1468-1474 (1989); Rossant, J., Neuton 2: 323-334 (1990)).

### V. Assessing Delivery of an Agent

In accordance with the present invention, the introduction of exogenous agents to cells for the screening and evaluation the agent's ability to silence the target gene, preferably, tetR-ODC, both in vitro and in vivo can in assessed utilizing various delivery methods. Exemplary methods are described below. The exogenous agent may be in a delivery complex, such as for example, coating with lipids or cell surface receptors or transfecting agents, encapsulation in biopolymers (e.g., poly-1 quadrature 4 N acetylglucosamine polysaccharide; see, U.S. Pat. No. 5,635,493), encapsulation in one or more lipid based formulations (e.g., liposomes), microparticles, or microcapsules; by administering it in linkage to a peptide or other ligand known to enter the nucleus; or by administering it in linkage to a ligand subject to receptor mediated endocytosis (see, e.g., Wu and Wu, J. Biol. Chem., 1987; 62:4429 4432), etc. Examples of one or more lipid based formulations that can be used are 1,2-dioleoyl-3-trimethylammonium-propane (DOTAP), 1,2-dioleoyl-3-(dimethylamino) propane (DODAP), 3β-[N-(N′,N′-dimethylaminoethane)-carbamoyl]cholesterol (DC-Chol) and cholesterol (Chol) (which can be purchased from Avanti Polar Lipids (Alabaster, Ala.)). Additional lipid based formulations that can be used are dioleoyl phosphatidylethanolamine (DOPE) and 1,2-distearoyl-sn-glycero-3-phosphoethanolamine-N-(carboxy(polyethylene glycol)2000) which can be purchased from Genzyme (Cambridge, Mass.). The compositions for the DOTAP, DODAP and DC-Chol-based lipid formulations can be DOTAP/DOPE/Chol/PEG-DSPE (40/39/20/1, molar %), DODAP/DSPC/Chol/PEG-DSPE (40/10/48/2, molar %), and DC-Chol/DOPE/DPPC/PEG-DSPE (40/20/39/1, molar %) respectively. For example, to prepare a lipid based formulation such as liposomes, lipids can be dissolved in tert-butanol at a final concentration of 10 mg/mL. siRNA can be dissolved in dH₂O and further mixed in an acidic citrate buffer. After brief heating in a 60° C. water bath, the lipid solution can be injected into the siRNA solution through a 28-gauge needle while the siRNA solution is under magnetic stirring to form emulsion. The resulting emulsion can be further buffered using a pH 7.4 phosphate buffered saline (PBS) at room temperature, and then subjected to diafiltration (Pellion ultrafiltraiton unit, MW 100K, PES membrane, Millipore) against PBS before concentration to the desired volume. The final lipid based formulation can have a siRNA concentration of 250 ug/mL. Such resulting liposomes may exhibit the follow characteristics: DODAP-based liposome (size=139 nm, PDI=0.09, zeta potential=−0.735 mV), DOTAP-based liposome (size=169 nm, PDI=0.17, zeta potential=4.32 mV), and DC-Chol-based liposome (size=166 nm, PDI=0.27, zeta potential=6.01 mV). Samples can be sterile filtrated through 0.22 μm syringe filters before further biological evaluation.

In another embodiment, an agent-delivery complex can be formed in which the ligand comprises a fusogenic viral peptide to disrupt endosomes, allowing the nucleic acid to avoid lysosomal degradation, or cationic 12 mer peptides, e.g., derived from antennapedia, that can be used to transfer therapeutic DNA into cells (Mi et al., Mol. Therapy, 2000; 2:339 47). In yet another embodiment, the nucleic acid can be targeted in vivo for cell specific uptake and expression, by targeting a specific receptor (see, e.g., PCT Publication Nos. WO 92/06180, WO 92/22635, WO 92/20316 and WO 93/14188).

### VI. Kits

The instant teachings also provide kits designed to facilitate the subject methods. Kits serve to expedite the performance of the disclosed methods by assembling two or more components required for carrying out certain methods. Kits can contain components in pre-measured unit amounts to minimize the need for measurements by end-users and can also include instructions for performing one or more of the disclosed methods. Typically, kit components are optimized to operate in conjunction with one another.

The disclosed kits may be used to identify, detect, and/or quantify exogenous agent delivery, including siRNA delivery, into a cell. In certain embodiments, kits comprising a tetR siRNA, and cells genetically altered to place the reporter gene under the control of the promoter. In a further embodiment, kits comprising a cell line engineered to respond to the delivery of the tetR-ODC siRNA with an increase of the reporter, preferably the lacZ reporter activity.

### EXPERIMENTAL

### Example 1

### TetR-ODC Construct Preparation and Determination of tetR-ODC Protein Stability

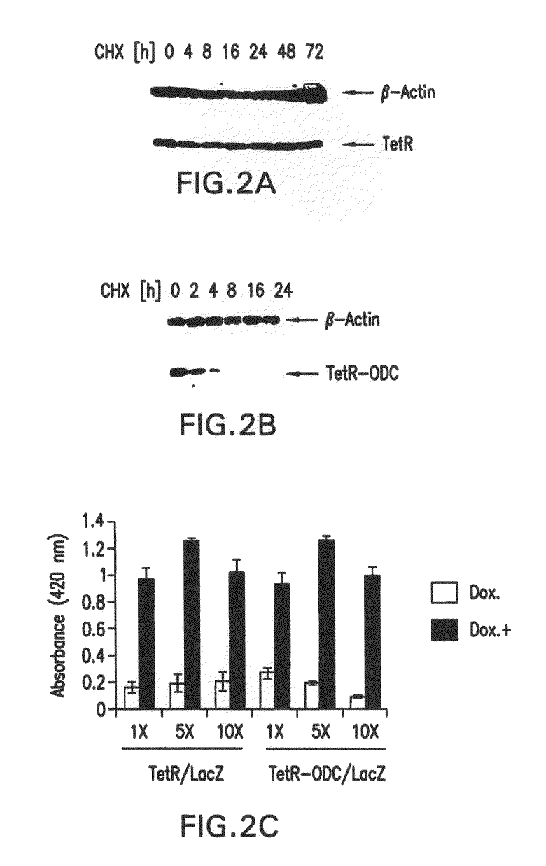

Unlike the small molecule inducer tetracycyline/doxycycline that triggers immediate onset of gene expression, the onset of tetR siRNA-induced reporter transcription will depend on the half-life of tetR because degradation of tetR protein is required to induce reporter transcription. To determine the half-life of tetR, we monitored the diminishing of tetR after blocking protein synthesis in an engineered tetR-expressing cell line, D54MG-tetR. The tetR protein was found to be extremely stable with a half-life far longer than 72 hours (FIG. 2A), indicating that a system based on the wild type tetR protein will not respond promptly to siRNA delivery. To shorten the half-life of tetR, we engineered a fusion protein with a protein degradation domain from ornithine decarboxylase (ODC) attached to the C-terminus of tetR (tetR-ODC). The tetR-ODC protein exhibited an intracellular half-life of 2 hours (FIG. 2B), indicating that the ODC-derived protein degradation domain is functional in mediating rapid protein degradation in the context of the fusion protein. To further determine whether tetR-ODC retains the ability to regulate the expression of tet-responsive promoters, we transfected H1299 cells using a tet-responsive lacZ reporter together with the wild type tetR or the tetR-ODC expression construct and monitored the transfected cells for their response to doxycycline treatment. Cells transfected with the wild type tetR or the tetR-ODC expression constructs exhibited similar levels of basal and doxycycline-induced beta-galactosidase activity (FIG. 2C), indicating that tetR-ODC retains the full activity of the wild type tetR.

A) D54MG cells that stably express the wild type tetR protein were treated with cycloheximide (CHX) for 0 to 72 hours. The cells were collected at indicated time points and the intracellular levels of tetR protein were determined by Western analysis. B) H1299 cells transfected with the tetR-ODC expression construct were treated with cycloheximide for 0 to 24 hours, and the protein level of tet-RODC was determined by Western analysis. C) H1299 cells were transfected with a tetracycline responsive LacZ reporter (LacZ) and a plasmid expressing the wild type tetR or the tetR-ODC protein at ratios of 1:1, 1:5, or 1:10 (LacZ reporter: tetR or tetR-ODC plasmid). 24 hours after transfection, the cells were switched to doxycycline containing medium (Dox+) or kept in regular medium without doxycyline (Dox −) for an additional 48 hours, and the lacZ reporter activity was determined using a beta-galactosidase assay kit. D) Coding sequence of the tetR-ODC protein. The TetR coding sequence (bold sequences) and the ODC coding sequence (italic sequences) were amplified by PCR and linked together via restriction enzyme digestion and ligation (see FIG. 1).

### Example 2

### siRNA Preparation

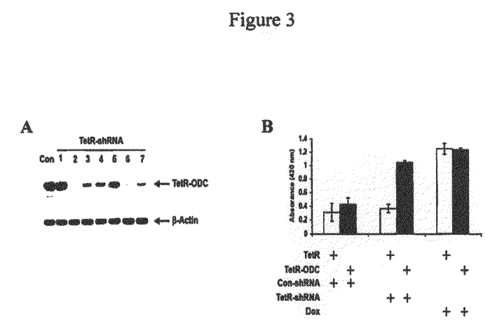

In addition to obtaining a destabilized tetR protein, the availability of a potent siRNA against tetR is another critical requirement for establishing a sensitive and robust assay for monitoring siRNA delivery. We have compared the characteristics of functional siRNA vs. functional shRNAs using data derived from a large set of shRNAs and siRNAs. It was found that functional shRNAs and siRNAs share extensive similarities in G/C preference along the duplex, suggesting that RNA duplexes derived from functional shRNAs are likely functional siRNAs. To identify potent siRNAs against tetR-ODC, we screened more than 20 shRNAs against tetR-ODC using a contransfection assay. After the primary screen and rigorous dose-response experiments, shRNA2 was found to be the most potent shRNA against tetR-ODC (FIG. 3A). Furthermore, shRNA2 triggered similar degrees of reporter activation as doxycycline treatment when transfected into cells (FIG. 3B), indicating that knockdown of tetR-ODC is indeed a valid alternative to doxycycline treatment for inducing the reporter activation.

A) Plasmids encoding a control shRNA (con) or different shRNAs designed to against the TetR-ODC protein (shRNA 1 to 7) were cotransfected into H1299 cells with the tetR-ODC expressing construct. 48 hours after transfection, cells were lysed and the amount of tetR-ODC protein was determined by western analysis. B) H1299 cells were transfected with a tet-responsive LacZ reporter and plasmids encoding wild type tetR (TetR) or the tetR-ODC (TetR-ODC) proteins, and plasmids encoding a control shRNAs (con-shRNA) or the TetR-shRNA2 (TetR-shRNA). The presence or absence of a particular plasmid was indicated as (+) or (−) respectively. Transfected cells were then cultured in the presence (+) or absence (−) of doxycycline containing for 48 hours and the activity of beta galactosidase in cells was determined using a beta galactosidase activity kit.

A) Plasmids encoding a control shRNA (con) or different shRNAs designed to against the TetR-ODC protein (shRNA 1 to 7) were cotransfected into H1299 cells with the tetR-ODC expressing construct. 48 hours after transfection, cells were lysed and the amount of tetR-ODC protein was determined by western analysis. B) H1299 cells were transfected with a tet-responsive LacZ reporter and plasmids encoding wild type tetR (TetR) or the tetR-ODC (TetR-ODC) proteins, and plasmids encoding a control shRNAs (con-shRNA) or the TetR-shRNA2 (TetR-shRNA). The presence or absence of a particular plasmid was indicated as (+) or (−) respectively. Transfected cells were then cultured in the presence (+) or absence (−) of doxycycline containing for 48 hours and the activity of beta galactosidase in cells was determined using a beta galactosidase activity kit.

SEQ ID NO: 3

| | |
|---|---|
| siRNA (tetR) sense: | 5′ GUUGCGUAUUGGAAGAUCA 3′ |

### Example 3

### Preparation of the Engineered Lacz Reporter Cell Lines that Express High Levels of Lacz Reporter Upon siRNA Delivery

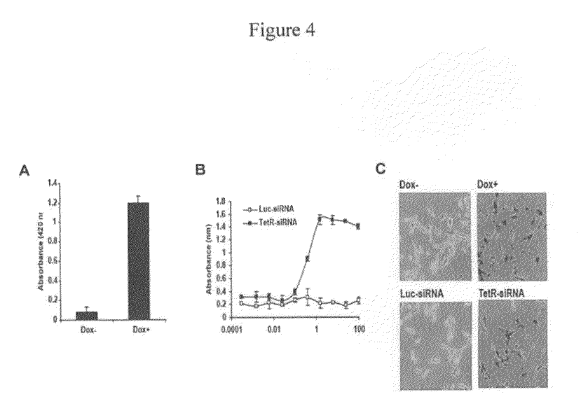

To create cancer cell lines that will respond to the delivery of siRNA with an increase of lacZ reporter activity, we first created MDA-MB435SLM-derived clones carrying the tetR-ODC expression cassette, then introduced a tet-responsive lacZ reporter into these cells. Upon screening a large number of stable clones for their response to doxycycline, several clones were found to exhibit minimal beta-galactosidase activity in the absence of doxycycline and a robust induction of beta-galactosidase activity in the presence of doxycycline (FIG. 4A). The ability of a tetR siRNA (siTetR) to induce reporter expression was also tested in these cell lines. The Lipofectamine-transfected siTetR caused a dose-dependent induction of beta-galactosidase expression with an estimated EC50 of 1 nM (FIG. 4B). In addition, X-gal staining of the MDA-tetR-ODC/LacZ cells under various treatments revealed that similar to doxycycline treatment, siTetR transfection also induced beta-galactosidase expression in the majority of cells. In contrast, cells transfected with the luciferase siRNA exhibited very low levels of staining (FIG. 4C). These results collectively suggest that a robust and tightly regulated reporter expression can be achieved in the MDA-tetR-ODC/LacZ cells upon the delivery of sitter.

A) An MDA-MB-435SLM-derived cell line with stably integrated tetR-ODC expression cassette and the tet-responsive lacZ reporter (MDA-tetR-ODC/LacZ) was cultured in the presence (Dox+) or absence (Dox−) of doxycycline for 72 hours, and the lacZ reporter activity was determined using the beta-galactosidase assay kit. B) The MDA-tetR-ODC/LacZ cells were transfected with different amounts of siRNA and a beta-galactosidase activity was determined using a beta-gal assay kit. C) The MDA-tetR-ODC/LacZ cells were cultured in the presence (Dox+) or absence of doxycycline (Dox−) for 72 hours (top panel) or transfected with a control siRNA (Luc-siRNA) or a tetR siRNA (TetR-siRNA) and cultured in the absence of doxycycline for 72 hours (bottom panel). The beta-galactosidase activity in these cells was determined by x-gal staining.

### Example 4

### Induction of Beta-Galactosidase Activity in Xenograft Tumors Using Doxycycline

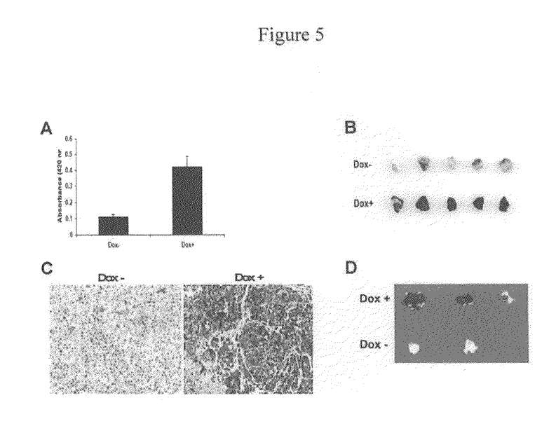

The robust response of the MDA-tetR-ODC/LacZ cells in vitro prompted us to further test the in vivo inducibility of the tetR-ODC system. The MDA-tetR-ODC/lacZ cells were used to created flank tumors and the tumor-bearing mice were fed with doxycycline for 5 days to induce the lacZ reporter expression. An apparent increase of beta-galactosidase activity was observed in tumor lysate from doxycycline-treated mice, which was further confirmed using whole-mount X-gal staining of the tumors (FIGS. 5A and 5B). Immunohistochemistry analysis of tumor sections from the doxycycline-treated mice revealed higher levels of beta-galactosidase expression in the entire section. Meanwhile, sections from the control tumors only had very weak staining (FIG. 5C). Since liver cancers are the primary focus of the siRNA therapeutic program in the near term, we further created orthotopic liver tumors by intrahepatic injection of the MDA-tetR-ODC/lacZ cells. Similar to the flank tumors, MDA-tetR-ODC/lacZ-derived orthotopic liver tumors also exhibited robust responses to doxycycline treatment (FIG. 5D). These results suggest that a robust induction of beta-galactosidase expression can be achieved in the MDA-tetR-ODC/LacZ-derived flank and orthotopic liver tumors. These tumor models are currently being used to evaluate siRNA deliver systems. Further effort is directed at establishing additional flank and liver tumor models containing the tetR-ODC system and exploring the possibility of creating transgenic mice with integrated tetR-ODC system. If created successfully, these transgenic mice will be valuable tools to assess functional siRNA delivery in the whole body of an animal.

Induction of beta-galactosidase activity in tumors. The MDA-MB-435SLM derived tetR-ODC/lacZ cells (MDA-tetR-ODC/LacZ) were inoculated into skid mice to establish flank tumors. Tumors from mice that fed with water (Dox−) or doxycycline (Dox+) for 7 days were collected and the beta-galactosidase expression in tumors were determined by A) beta-galactosidase assay using tumor lysate, B) x-gal staining of tumor tissues, and C) IHC analysis of tumor sections using a beta-galactosidase specific antibody. D) The MDA-tetR-ODC/LacZ cells were used to establish orthotopic liver tumors via intrahepatic injection. Liver tumors from mice treated with water (Dox−) or doxycycline (Dox+) for 7 days were isolated, and the beta-galactosidase expression in tumors were determined using x-gal staining.

The above-described exemplary embodiments are intended to be illustrative in all respects, rather than restrictive, of the present invention. Thus, the present invention is capable of implementation in many variations and modifications that can be derived from the description herein by a person skilled in the art. All such variations and modifications are considered to be within the scope and spirit of the present invention as defined by the following claims.

### Example 5

### TetR-Based Tumor Model Utilizing A Luciferase Reporter and Bioluminescence Imaging

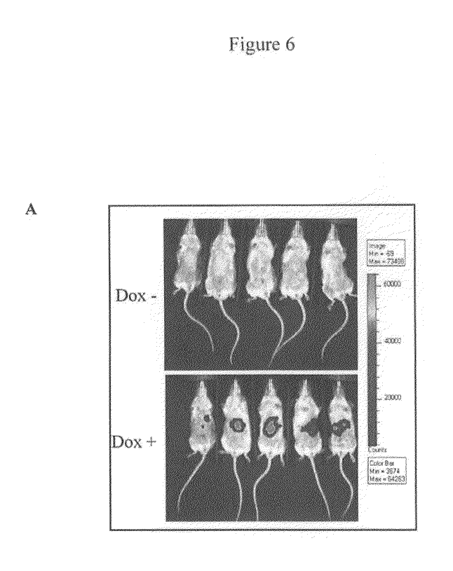

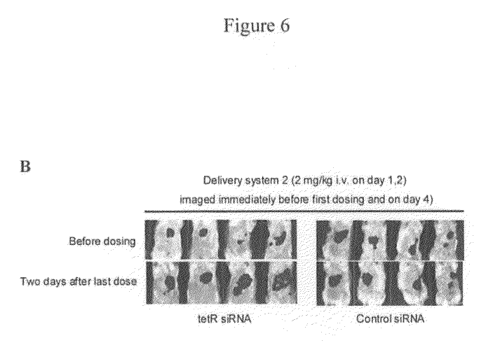

A MDA-tetR-ODC/Luc cell line was created from MDA-MB435-SLM-derived cell line with a stably integrated tetR-ODC expression cassette and a tet-responsive firefly luciferase reporter. FIG. 6A). The MDA-tetR-ODC/Luc cells were inoculated into skid mice to establish liver tumors. The mice were imaged before doxycycline treatment (top panel of FIG. 6A) or 4 days after doxycycline treatment (bottom panel of FIG. 6A). Liver tumors that are derived from the MDA-tetR-ODC/Luc cells, exhibited a more than 100-fold increase of luminescent signal in doxycycline-treated mice compared to the untreated mice, indicating that the MDA-tetR-ODC/Luc-derived tumors could be very sensitive to siRNA delivery in vivo. FIG. 6B) Monitoring target knockdown in the MDA-tetR-ODC/Luc cell-derived liver tumors using imaging. The MDA-tetR-ODC/Luc cells were inoculated into skid mice to establish liver tumors. Mice bearing the MDA-tetR-ODC/Luc liver tumors were injected at 2 mg siRNA/kg on day 1 and 2 with a lipid based delivery formulation containing the control siRNA or the tetR siRNA. Mice were imaged before dosing and 2 days after the last dose. Lipid-mediate delivery of the tetR siRNA into the MDA-tetR-ODC/Luc cells triggered a dramatic increase of luciferase activity in these cells, suggesting that this cell line is highly responsive to intracellular siRNA delivery (FIG. 6B). The mice were imaged before doxycycline treatment (top panel of FIG. 6B) or 4 days after doxycycline treatment (bottom panel of FIG. 6B).

What is claimed is:

1. A method of analyzing the delivery of an exogenous agent to a tissue comprising: (a) providing a tissue that expresses a fusion protein, wherein said fusion protein has a half-life of no more than about 72 hours; (b) contacting said tissue with the exogenous agent; (c) analyzing a sample from the tissue, thereby providing analysis of the delivery of the exogenous agent to the tissue.

2. The method of claim 1, wherein said half life is no more than about 48 hours.

3. The method of claim 1, wherein said half life is no more than about 12 hours.

4. The method of claim 1, 2 or 3, wherein said cells contain the nucleic acid that encodes a detectable protein that is operably linked to an inducible promoter.

5. The method of claim 1, 2 or 3 wherein said fusion protein comprises a transcription repressor.

6. The method of claim 5 wherein said transcription repressor is tetracycline (tet) repressor protein.

7. The method of claim 5 wherein said transcription repressor is the lac repressor.

8. The method of claim 6 wherein said tet repressor protein further comprises a PEST sequence.

9. The method of claim 1, 2 or 3, wherein said exogenous agent is a nucleic acid.

10. The method of claim 9 wherein said agent is a nucleic acid molecule selected from the group consisting of a siRNA, microRNA, ribozyme, and an antisense.

11. The method of claim 8, wherein said PEST sequence is a PEST sequence-containing portion of a C-terminus of murine ornithine decarboxylase (MODC).

12. The method of claim 6, wherein said detectable protein is selected from the group consisting of humanized renilla green fluorescent protein (hrGFP), enhanced green fluorescent protein (eGFP), enhanced blue florescent protein (eBFP), enhanced blue fluorescent protein (eBFP), enhanced cyan fluorescent protein (eCCFP), enhance yellow fluorescent, red fluorescent protein (RFP or DsRed) firefly luciferase, and renilla luciferase.

13. The method of claim 6 wherein said tetR repressor protein comprises the sequence of SEQ ID No: 2.

14. The method of claim 1, wherein said cells are cancer cells.

15. The method of claim 10, wherein said siRNA agent comprises an antisense strand of 17-25 nucleotides complementary to a sense strand, wherein said sense strand is selected from 17-25 continuous nucleotides of a nucleic acid sequence of SEQ ID NO: 2.

16. The method of claim 1 wherein said fusion protein having a half life of about 4 hours.

17. The method of claim 1 wherein said fusion protein having a half life of about 2 hours.

18. The method of claims 1, wherein said exogenous agent is complexed with a delivery agent.

19. The method of claim 1, wherein said detectable marker is lacZ and said inducible promoter is a tet-responsive promoter.

20. A method of analyzing the delivery of an exogenous agent to a tissue comprising: (a) providing a tissue having the tet fusion protein which further comprises a PEST sequence, wherein said PEST sequence is a PEST sequence-containing portion of a C-terminus of murine ornithine decarboxylase (MODC); (b) contacting said tissue with the exogenous agent; (c) analyzing a sample from the tissue, thereby providing analysis of the delivery of the exogenous agent to the tissue.

21. The method of claim 20 wherein said agent is a nucleic acid molecule selected from the group consisting of a siRNA, microRNA, ribozyme, and an antisense.

22. The method of claim 20 wherein said tissue is subjected to one or more physical or chemical treatments.

23. The method of claim 20 wherein the tissue is an animal tissue.

24. The method of claim 20 wherein the tissue is diseased or injured.

25. The method of claim 20 wherein the animal tissue is cancerous.

26. The method of claim 1, wherein said exogenous agent is a siRNA which silences the tet repressor gene.

27. The method of claim 21, wherein said siRNA agent comprises an antisense strand of 17-25 nucleotides complementary to a sense strand, wherein said sense strand is selected from 17-25 continuous nucleotides of a nucleic acid sequence of SEQ ID NO: 2.

28. The method of claim 20, wherein said fusion protein having a half life of no more than about seventy two hours.

29. The method of claim 20, wherein said fusion protein having a half life of about no more than 48 hours.

30. The method of claim 20, wherein said fusion protein having a half life of about no more than 12 hours.

31. The method of claims 20, wherein said agent is complexed with a delivery agent.

32. The method of claim 20, wherein said detectable marker is lacZ and said inducible promoter is a tet-responsive promoter.

33. A transgenic non-human animal comprising a recombinant nucleic acid molecule stably integrated into the genome of said animal, said molecule encoding a tet repressor fusion protein further comprises a PEST sequence, wherein said PEST sequence is a PEST sequence-containing portion of a C-terminus of murine ornithine decarboxylase (MODC).

34. A non-human animal of claim 33, wherein said detectable marker is selected from the group consisting of humanized renilla green fluorescent protein (hrGFP), enhanced green fluorescent protein (eGFP), enhanced blue florescent protein (eBFP), enhanced blue fluorescent protein (eBFP), enhanced cyan fluorescent protein (eCCFP), enhance yellow fluorescent, red fluorescent protein (RFP or DsRed), beta-galactosidase, and luciferase.

35. A method of analyzing the delivery of an exogenous agent to a tissue comprising: (a) providing a non-human transgenic non-human animal comprising a recombinant nucleic acid molecule stably integrated into the genome of said animal, said molecule encoding a tet fusion protein further comprises a PEST sequence, wherein said PEST sequence is a PEST sequence-containing portion of a C-terminus of murine ornithine decarboxylase (MODC); (b) contacting said non-human animal with the exogenous agent; (c) analyzing a sample from the tissue of the non-human animal, thereby providing analysis of the delivery of the exogenous agent to the tissue.

36. The method of claim 35, wherein said detectable marker is beta-galactosidase.

37. A method of analyzing the delivery of an exogenous agent to a tissue comprising: (a) providing a non-human transgenic non-human animal comprising a recombinant nucleic acid molecule stably integrated into the genome of said animal, said molecule encoding a tet fusion protein further comprises a PEST sequence, wherein said PEST sequence is a PEST sequence-containing portion of a C-terminus of murine ornithine decarboxylase (MODC); (b) contacting said non-human animal with the exogenous agent; (c) non-invasively detecting the delivery of the exogenous agent to the tissue.

38. The method of claim 37, wherein said non-invasive detection is by imaging.

39. The method of claim 39, wherein said imaging is bioluminescence imaging.

40. The method of claim 37, 38 or 39, wherein said detectable marker is luciferase.

41. The method of claim 1, 20, 35 or 37 wherein the exogenous agent is included in a lipid based formulation.

42 The method of claim 41, wherein the lipid based formulation is 1,2-dioleoyl-3-trimethylammonium-propane, 1,2-dioleoyl-3-(dimethylamino) propane, 3β-[N-(N′,N′-dimethylaminoethane)-carbamoyl]cholesterol, cholesterol, dioleoyl phosphatidylethanolamine or 1,2-distearoyl-sn-glycero-3-phosphoethanolamine-N-(carboxy(polyethylene glycol)2000).
